(19) United States
(12) Patent Application Publication
(10) Pub. No.: US 2008/0319520 A1
(43) Pub. Date: Dec. 25, 2008

(54) Endocardial Lead

(76) Inventor: Rolf Hill, Jarfalla (SE)

Correspondence Address: SCHIFF HARDIN, LLP;PATENT DEPARTMENT, 6600 SEARS TOWER, CHICAGO, IL, 60606-6473, US

(21) Appl. No.: 12/093,858

(22) PCT Filed: Nov. 30, 2005
(86) PCT No.: PCT/SE2005/001810

§ 371 Date: May 15, 2008

(51) Int. Cl. A61N 1/05

(52) U.S. Cl. 607/127

(57) ABSTRACT

A device terminating a distal end of a cardiac stimulator lead for transmitting electrical signals from a pacemaker to a location within a heart for stimulating cardiac tissue of the heart said device comprising an electrically conductive helix configured to interact with said cardiac tissue by attachment to said cardiac tissue with a rotating screw motion, said helix having a plurality of helical flights respectively oriented at a screw pitch a tube containing said helix and allowing rotation of said helix in an interior of said tube said tube having an interior post that protrudes between adjacent flights of said helix a rotatable shaft on which said helix is mounted, rotation of said shaft in respective directions causing outward and inward screwing motion of said helix relative to said tube by engagement of said post with said flights of said helix, and said shaft having a stop surface thereon that, when said stop surface reaches said post, interrupts said screw rotation of said helix by abutment of the stop surface against said post, said stop surface being oriented in a plane that deviates less than 450 relative to a radial direction fo said shaft and relative to a direction perpendicular to said screw pitch.

### BACKGROUND OF THE INVENTION

1. Field of the Invention

The invention relates to a device for extending and retracting of a helix from a distal end of a cardiac stimulator lead, of the type used for transmitting electrical signals from a pacemaker to a position inside a heart for stimulating the heart activity, wherein the helix is used for attaching the cardiac stimulator lead to a heart wall by screw rotating the helix into the heart wall, of the type having a tip tube, with an interior post which that protrudes between adjacent coils of the helix, and a shaft on which the helix is mounted, such that when rotating the shaft by means of a rotary member, the helix as well as the shaft will be screw rotated outward or inward in the tip tube by engagement between the helix and the post.

The invention also relates to a cardiac stimulator lead comprising such a device.

2. Description of the Prior Art

When screw rotating a helix of a cardiac stimulator lead, for extending it from the distal end of the tip tube to attach the cardiac stimulator lead to the heart wall, it is essential that the helix will not be extended too far out from the tip tube since the helix then may penetrate through the heart wall. It is also essential that the helix may be retracted again if, for any reason, it is desirable to detach the cardiac stimulator lead if necessary. This may be difficult or even impossible if the helix has been extended so far such that the helix coils have been disengaged from the post. It is also a risk that the post and the shaft can get jammed together, if the helix is extended so far such that the post and the shaft will meet, which will make it difficult or impossible to retract the helix.

In the prior art it is known to arrange a separate stop member in the proximal end of the shaft, which will restrict the maximum extension of the shaft and helix by abutment against a holder holding the rotary shaft. However, the developments goes towards thinner cardiac stimulator leads, preferably less than 3 mm outside diameter. It is then a need for reducing the number of small components, such as the stop member, since these small components will be very difficult to handle during assembling in production and also expensive to produce.

### SUMMARY OF THE INVENTION

An object of the invention to eliminate or reduce the drawbacks according to the prior art and provide a device, for extending and retracting of a helix from a distal end of a cardiac stimulator lead, which is able to restrict the extension of the helix without the risk of the helix to get jammed or without the need for any separate stop member.

The invention is thus based on the recognition that this object may be achieved by a shaft, for holding the helix, which is formed with a stop surface.

Within this general inventive idea, the invention may be implemented in many different ways. However, the stop surface must be oriented such that its extension has at least one component perpendicular to the direction in which the shaft is moved when rotated for extending the helix. Also, the shape of the stop surface may be any arbitrary, appropriate shape being able to function as a stop surface, e.g. a curved or a plane surface.

In an embodiment of the invention, the stop surface is a plane surface and oriented in a plane which is parallel to the radial and longitudinal directions of the shaft. However, the invention is not restricted to this orientation. The stop surface could for example also be oriented in a plane which is parallel to the radial direction but has a small angle, corresponding to the pitch angle of the helix, to the longitudinal direction, such that the stop surface is slightly turned towards the tip of the helix, i.e. in the direction in which the stop surface is moved when screw rotating the helix to an extended position. In reality the actual stop surface may deviate a rather great deal from the latter orientation. However, in order to eliminate the risk for jamming between the stop surface and the post, the deviation in relation to the orientation perpendicular to the pitch angle, should be less than 450 and preferably less than 300 in relation to the radial direction as well as the direction perpendicular to the screw pitch of the helix.

Within the scope of the invention, the device may be formed in many different ways. For example, in the described embodiment the stop surface is formed on the shaft in the area between the two most proximal coils of the helix. The helix is attached to the shaft by way of a groove, which is formed in an enlarged portion of the shaft and which has a shape corresponding to the helix. However, both of these features as well as many others, may be performed in different ways as will be evident for anyone skilled in the art.

As used herein, the term “distal” relates to a position or a direction closer to the outermost end of the cardiac stimulator lead, which is intended to be attached to the heart wall, whereas the term proximal relates to a position or a direction further away from the outermost end of the cardiac stimulator lead.

### BRIEF DESCRIPTION OF THE DRAWINGS

FIG. 1 is a perspective view, partly in section, of a tip tube, located at the distal end of a cardiac stimulator lead, having the helix in a retracted position, in accordance with the present invention.

FIG. 2 is a perspective view of the embodiment of FIG. 1, with the helix in an extended position.

FIG. 3 is a perspective view of the assembled helix and shaft mounted in a holder.

FIG. 4 is a perspective view of the shaft.

### DESCRIPTION OF THE PREFERRED EMBODIMENTS

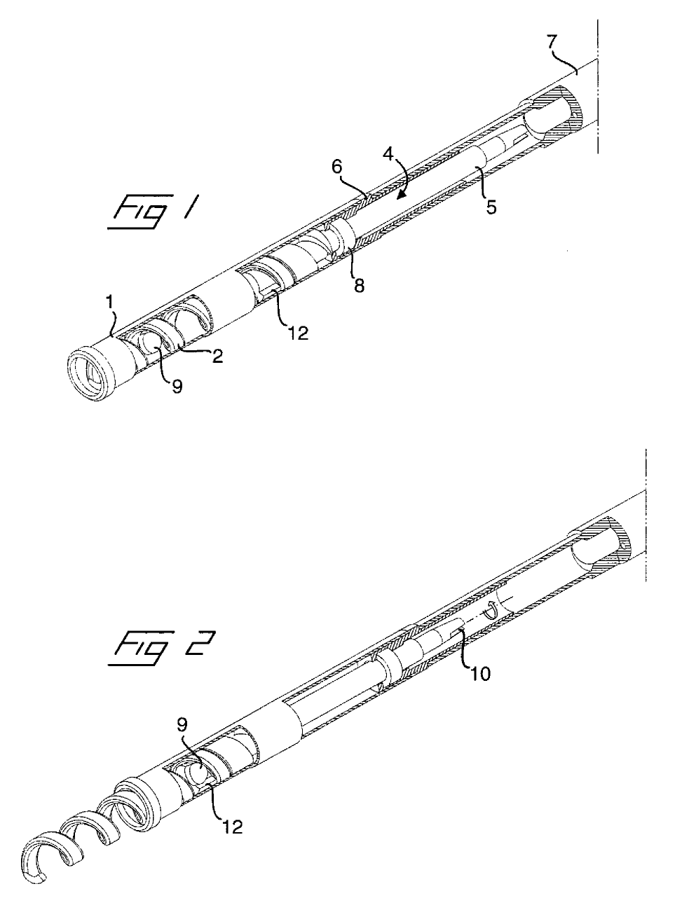

Reference is first made to FIGS. 1 and 2, in which a distal end of a cardiac stimulator lead is shown in a partly cut through perspective view. The cardiac stimulator lead comprises a tip tube 1 in which a helix 2 is accommodated. The helix has a cork screw form and is in its proximal end attached to an enlarged helix holding portion 3 in the distal end of a shaft 4. The proximal portion of the shaft functions as a guiding portion 57 which is rotating and sliding supported in a holder 6 mounted to the proximal end of the tip tube. Besides a guiding function for the shaft 4, the holder 6 is adapted to provide a sealing around the shaft to prevent fluids from coming in from the tip tube into the holder and further into the cardiac stimulator lead, which is indicated at 7. The sealing effect is accomplished by a seal 8 between the holder and the shaft.

A post 9 protrudes from the inner surface of the tip tube 1 into a gap between two adjacent coils of the helix 2. By engagement between the helix coils and the post, the helix 2 and the holder 4 is caused to be extended or retracted when rotating the holder. This is illustrated in FIG. 2 where the helix 2 is extended out from the distal end of the tip tube 1 until the post 9 abuts against a stop surface 12. The rotation of the shaft 4 may be performed by any suitable means, such as a flexible torque transferring wire (not shown), which can be inserted through the cardiac stimulator lead and be moved into engagement with an engagement formation 10 in the proximal end of the shaft 4, as is best seen in FIG. 4.

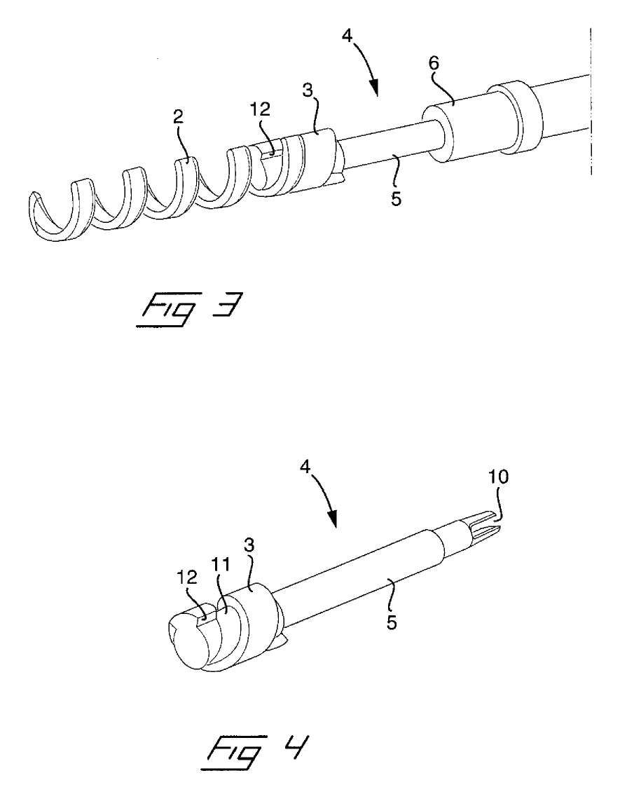

Now reference is made to FIGS. 3 and 4, wherein FIG. 3 shows the assembled helix 2 and shaft 4 inserted with the guide portion 5 of the shaft into the holder 6. The helix is attached to the shaft by way of a groove 11, as is best seen in FIG. 4 where the shaft 4 is illustrated separately. The groove 11 has a shape that essentially corresponds to the shape of the helix, such that a few coils of the helix may be threaded onto the shaft as is shown in FIG. 3. By removing a portion of the enlarged helix holding portion 3, a stop surface 12 is formed between the two most proximal coils of the helix. The stop surface, in the embodiment illustrated, is oriented in a plane which is parallel to the radial direction as well as the longitudinal direction of the shaft. I.e. the stop surface is oriented in a radial plane of the shaft.

In the view of FIG. 2, the helix is shown in an extended position in which the helix is extended out from the distal end of the tip tube. In this position, the stop surface 12 is in abutment with the post 9, which is positioned on the inner surface of the wall of the cut away tip tube 1, by screw rotation of the shaft 4. Accordingly, the helix is in this position restricted from extending any further out from the tip tube, however, without any risk for jamming of the post 9 between the helix (2) and the outer end face of the helix holding portion 3.

Although modifications and changes may be suggested by those skilled in the art, it is the intention of the inventor to embody within the patent warranted hereon all changes and modifications as reasonably and properly come within the scope of his contribution to the art.

I claim as my invention:

1-7. (canceled)

8. A device terminating a distal end of a cardiac stimulator lead for transmitting electrical signals from a pacemaker to a location within a heart for stimulating cardiac tissue of the heart, said device comprising:
an electrically conductive helix configured to interact with said cardiac tissue by attachment to said cardiac tissue with a rotating screw motion, said helix having a plurality of helical flights respectively oriented at a screw pitch;
a tube containing said helix and allowing rotation of said helix in an interior of said tube;
said tube having an interior post that protrudes between adjacent flights of said helix;
a rotatable shaft on which said helix is mounted, rotation of said shaft in respective directions causing outward and inward screwing motion of said helix relative to said tube by engagement of said post with said flights of said helix; and
said shaft having a stop surface thereon that, when said stop surface reaches said post, interrupts said screw rotation of said helix by abutment of the stop surface against said post, said stop surface being oriented in a plane that deviates less than 45° relative to a radial direction fo said shaft and relative to a direction perpendicular to said screw pitch.

9. A device as claimed in claim 8 wherein said stop surface is oriented in a plane that is parallel to the radial direction and to the direction perpendicular to the screw pitch.

10. A device as claimed in claim 8 wherein said stop surface is oriented in a plane parallel to said radial direction and parallel to a longitudinal direction of said tube.

11. A device as claimed in claim 8 wherein said stop surface is oriented in a plane that deviates by less than 30° relative to said radial direction and relative to said direction perpendicular to said screw pitch.

12. A cardiac stimulator lead comprising:
an insulating sleeve carrying at least one electrical conductor therein and having a proximal end configured for connection to a cardiac stimulator, and having a distal end; and
a device terminating said distal end of said cardiac stimulator lead for transmitting electrical signals from said conductor to a location within a heart for stimulating cardiac tissue of the heart, said device comprising an electrically conductive helix connected to said conductor and configured to interact with said cardiac tissue by attachment to said cardiac tissue with a rotating screw motion, said helix having a plurality of helical flights respectively oriented at a screw pitch, a tube containing said helix and allowing rotation of said helix in an interior of said tube, said tube having an interior post that protrudes between adjacent flights of said helix, a rotatable shaft on which said helix is mounted, rotation of said shaft in respective directions causing outward and inward screwing motion of said helix relative to said tube by engagement of said post with said flights of said helix, and said shaft having a stop surface thereon that, when said stop surface reaches said post, interrupts said screw rotation of said helix by abutment of the stop surface against said post, said stop surface being oriented in a plane that deviates less than 45° relative to a radial direction fo said shaft and relative to a direction perpendicular to said screw pitch.
